(19) United States
(12) Patent Application Publication
(10) Pub. No.: US 2018/0325752 A1
(43) Pub. Date: Nov. 15, 2018

(54) PROTECTIVE BARRIER SYSTEMS

(71) Applicants: Kendric Holloway, Country Club Hills, IL (US); Dorothy M. Holton, Country Club Hills, IL (US)

(72) Inventors: Kendric Holloway, Country Club Hills, IL (US); Dorothy M. Holton, Country Club Hills, IL (US)

(21) Appl. No.: 15/590,957

(22) Filed: May 9, 2017

(51) Int. Cl. A61F 13/70; A61F 13/82; A41B 9/12; A61F 13/537; A41B 9/00

(52) U.S. Cl. CPC A61F 13/70; A61F 13/82; A41B 2300/32; A61F 13/537; A41B 9/004; A41B 9/12

(57) ABSTRACT

A protective barrier system includes a protective barrier assembly having a groin covering panel including an aperture, a securing strap, and fastening members. The groin covering panel is formed of flexible material with leg openings, and is of a size and shape configured to cover a pelvic region of a user-wearer. The securing strap when fastened to the groin covering panel creates the leg openings which are configured to receive legs of the user-wearer and secure the groin covering panel to the user-wearer. The protective barrier system provides an absorbent article and protective barrier for absorbing excess fluids during the sexual activities.

### CROSS REFERENCE TO RELATED APPLICATION

The present application is related to and claims priority to U.S. Provisional patent application Ser. No. 29/505,977 filed May 9, 2016, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of sexual intercourse protection and hygiene products and more specifically relates to a protective barrier system.

### 2. Description of Related Art

Various protective devices are used to avoid contact with blood, vaginal fluid, semen or other contaminant agents (like skin, hair and shared objects) during sexual activity. Sexual activity using these devices is called protected sex. Engaging in sexual activity is primarily for pleasure. A need exists for a protective device for staying clean while engaging in sexual activities and avoid excess fluids and reduce mess. A suitable solution is desired.

U.S. Pat. No. 5,749,862 to Kai-Ming Lau relates to a condom improving device. The described condom improving device includes a condom improving device in the form of an elastic sheet having an aperture with a cross-sectional area of from 12.5 mm² to 707 mm², the aperture being adapted to receive a condom and the perimeter thereof acting to hold the rim of the condom on the body of a user.

### BRIEF SUMMARY OF THE INVENTION

In view of the foregoing disadvantages inherent in the known sexual intercourse protection art, the present disclosure provides a novel protective barrier system. The general purpose of the present disclosure, which will be described subsequently in greater detail, is to provide a protective barrier able to absorb any excess fluids during sexual activities.

A protective barrier system is disclosed herein. The protective barrier system includes a protective barrier assembly having a groin covering panel including an aperture, a securing strap, and fastening members. The protective barrier system comprises the protective barrier assembly. The protective barrier assembly comprises the groin covering panel, the securing strap, and a plurality of fastening members in functional combination. The groin covering panel is formed of flexible material with leg openings, and is of a size and shape configured to cover a pelvic region of a user-wearer. The securing strap when fastened to the groin covering panel creates the leg openings which are configured to receive legs of the user-wearer and secure the groin covering panel to the user-wearer. The groin covering panel comprises an absorbent planar sheet. The groin covering panel comprises the aperture configured to allow genitals to be accessed for sexual activities.

The securing strap may be fastened to the groin covering panel via fasteners. The fasteners may include hook and loop fasteners or other suitable fastening means. The protective barrier system provides an absorbent article and protective barrier for absorbing excess fluids during the sexual activities.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a protective barrier systems, constructed and operative according to the teachings of the present disclosure.

FIG. 1 is a perspective view of the protective barrier system during an ‘in-use’ condition, according to an embodiment of the disclosure.

FIG. 2 is a perspective view of the protective barrier system of FIG. 1, according to an embodiment of the present disclosure.

FIG. 3 is a perspective view of the protective barrier system of FIG. 1, according to an embodiment of the present disclosure.

FIG. 4 is a perspective view of the protective barrier system of FIG. 1, according to an embodiment of the present disclosure.

FIG. 5 is a perspective view of the protective barrier system of FIG. 1, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to sexual intercourse protection and more particularly to a protective barrier systems as used to improve the protective barrier system.

Generally, the protective barrier system provides an absorbent article for reducing ‘mess’ during sexual activities. The protective barrier system includes a groin covering panel with a removable securing strap to maximize user-wearer security and comfort. The protective barrier system's main objective is to absorb any excess fluids that may be unwanted and to make sure that a user-wearer remains clean while engaging in sexual activities. The device may comprise soft yet durable material on the inside and out, to be as comfortable as possible when it is in use. The removable securing straps provide support so that a user-wearer may maneuver in virtually any way they please and still remain covered. If the securing straps are too restricting they may easily be detached. Any kind of liquids or materials that the user-wearer would like to keep off of their pubic area will be ‘intercepted’ (isolated from) by the protective barrier system. The aperture located on the groin covering panel of the protective barrier system is also shaped so that any area of a woman's vagina is accessible. It all has elasticity to ensure that most any size penis will fit.

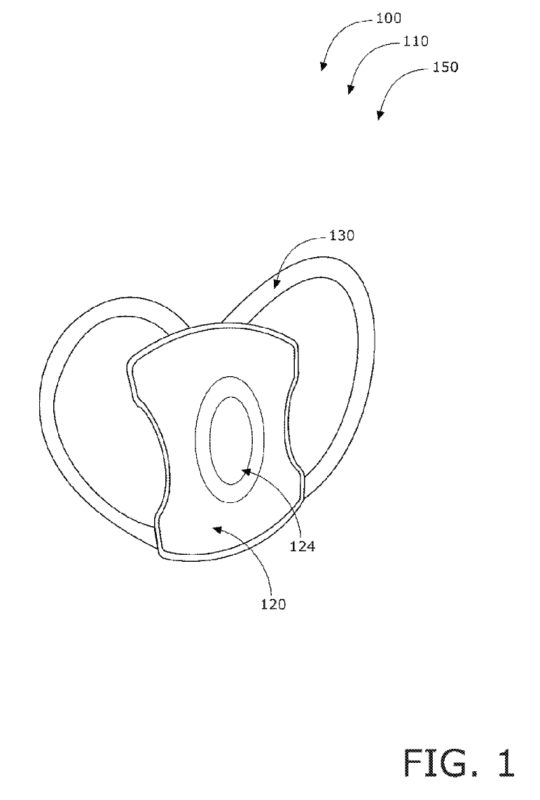

Referring now more specifically to the drawings by numerals of reference, there is shown in FIGS. 1-5, various views of a protective barrier system 100. FIG. 1 shows a protective barrier system 100 during an ‘in-use’ condition 150, according to an embodiment of the present disclosure. As illustrated, the protective barrier system 100 may include a protective barrier assembly 110 having a groin covering panel 120 including an aperture 124, a securing strap 130, and fastening members 138. The protective barrier system 100 comprises the protective barrier assembly 110. The protective barrier assembly 110 may comprise the groin covering panel 120, the securing strap 130, and the fastening members 138 in functional combination. The groin covering panel 120 is formed of flexible material with leg openings, and is of a size and shape configured to cover a pelvic region of a user-wearer.

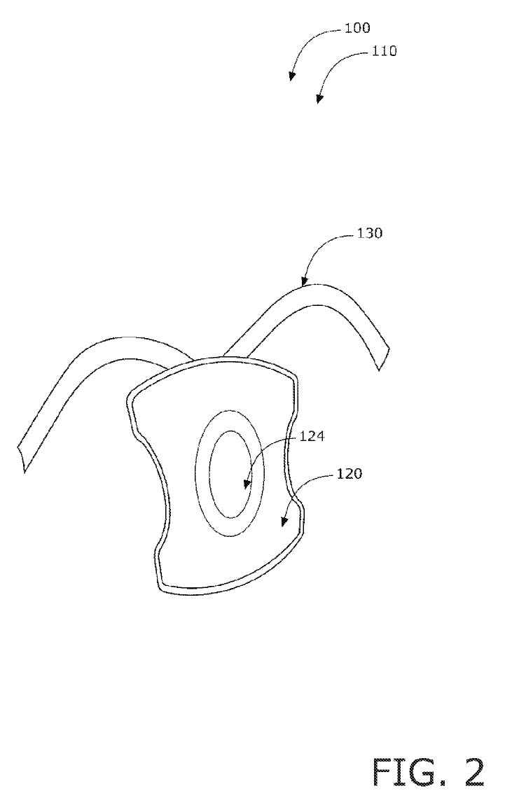

FIG. 2 shows a perspective view of the protective barrier system 100 of FIG. 1, according to an embodiment of the present disclosure. As above, the protective barrier system 100 may include a groin covering panel 120 including an aperture 124, a securing strap 130, and fastening members 138. In a preferred embodiment, the groin covering panel 120 comprises a substantially rectangular profile with contoured edges which are configured to contour to legs of the user-wearer. The groin covering panel 120 may comprise a flexible planar sheet comprising at least one wicking material for absorbing excess fluids. The groin covering panel 120 comprises the aperture 124 configured to allow genitals to be accessed for sexual activities. The aperture 124 may comprise an elastic collar. The aperture 124 is located at a center of the groin covering panel 120.

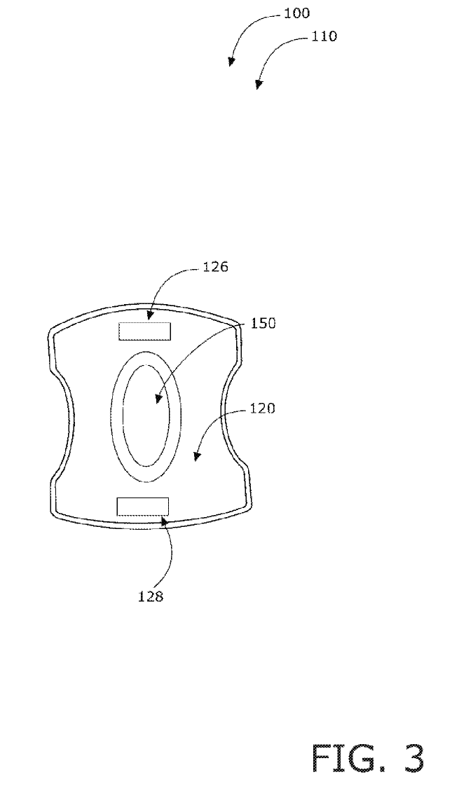

FIG. 3 shows a perspective view of the protective barrier system 100 of FIG. 1, according to an embodiment of the present disclosure. As above, the protective barrier system 100 may include a groin covering panel 120 including an aperture 124, a securing strap 130, and fastening members 138. In a preferred embodiment, the fastening members 138 may comprise hook and loop fasteners; alternate embodiments may use suitably equivalent fastening means. The securing strap 130 may comprise fastening members 138 located at a first-end, a middle, and a second-end of the securing strap 130. The groin covering panel 120 comprises a first-fastener 126 and a second-fastener 128 located on a top portion and a bottom portion, respectively of the groin covering panel 120. The first-fastener 126 and the second-fastener 128 are located on the groin covering panel 120 comprise female-fasteners of the hook and loop fasteners. The first-fastener 126 and the second-fastener 128 are located on a back side of the groin covering panel 120. The back side of the groin covering panel 120 is configured to rest against a pelvic region of the user-wearer.

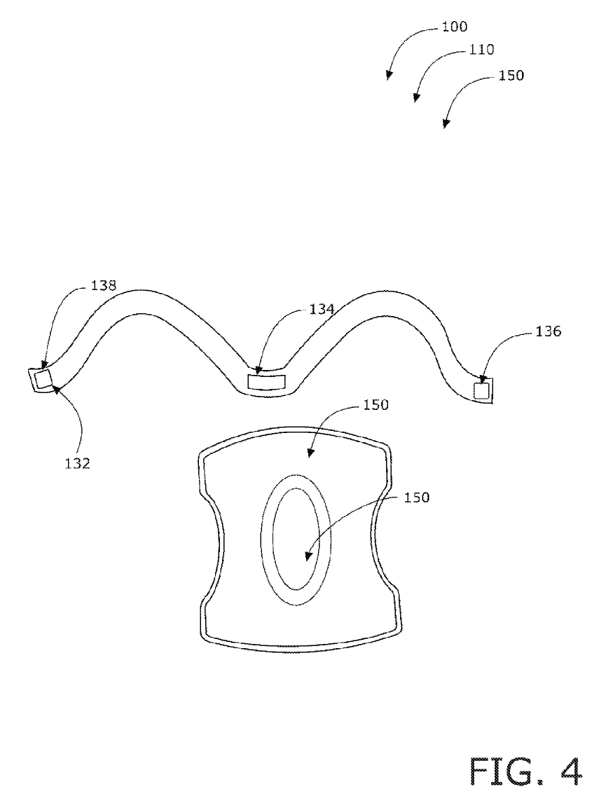

FIG. 4 shows a perspective view of the protective barrier system 100 of FIG. 1, according to an embodiment of the present disclosure. As above, the protective barrier system 100 may include a groin covering panel 120 including an aperture 124, a securing strap 130, and fastening members 138. Leg openings are formed by fastening a middle-fastener 134 of the securing strap 130 to the first-fastener 126 and fastening a first-end-fastener 132 of the securing strap 130 and a second-end fastener 136 of the securing strap 130 on opposing sides of the second-fastener 128 located at the bottom portion of the groin covering panel 120. The first-end-fastener 132, the middle-fastener 134, and the second-end fastener 136 of the securing strap 130 comprise male-fasteners of the hook and loop fasteners for mating with the female-fasteners located on the groin covering panel 120. The first-end-fastener 132 and the second-end fastener 136 comprise a smaller surface area than the middle-fastener 134, the first-fastener 126, and the second-fastener 128. The second-fastener 128 comprises a surface area which is configured to receive both the first-end-fastener 132 and the second-end fastener 136.

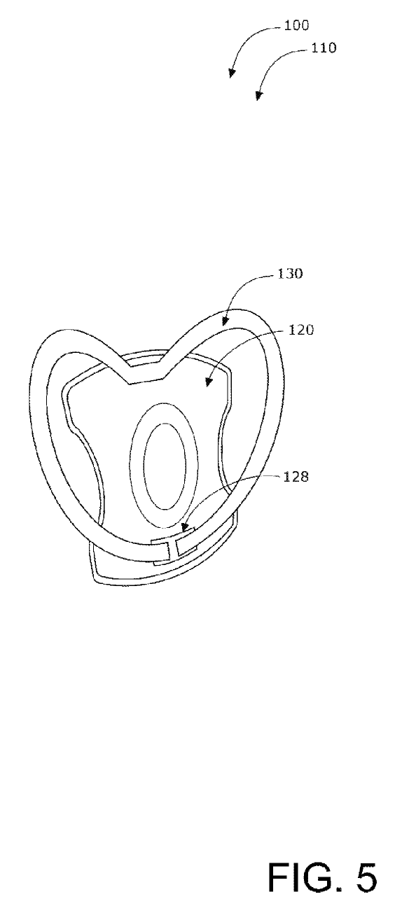

FIG. 5 shows a perspective view of the protective barrier system 100 of FIG. 1, according to an embodiment of the present disclosure. As above, the protective barrier system 100 may include a groin covering panel 120 and a securing strap 130 and fastening members 138. The securing strap 130 when fastened to the groin covering panel 120 creates the leg openings which are configured to receive legs of the user-wearer and secure the groin covering panel 120 to the user-wearer. The securing strap 130 may be elasticized to accommodate the size of the user-wearer. The protective barrier system 100 provides an absorbent article and protective barrier for absorbing excess fluids during the sexual activities.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by letters patent is set forth in the appended claims:

1. A protective barrier system comprising:
a protective barrier assembly including;
a groin covering panel;
an aperture;
a securing strap; and
fastening members;
wherein said protective barrier system comprises said protective barrier assembly;
wherein said protective barrier assembly comprises said groin covering panel, said securing strap, and said fastening members in functional combination;
wherein said groin covering panel is formed of flexible material with leg openings, and is of a size and shape configured to cover a pelvic region of a user-wearer;
wherein said groin covering panel comprises said aperture configured to allow genitals to be accessed for sexual activities;
wherein said securing strap comprises said fastening members located at a first-end, a middle, and a second-end of said securing strap;
wherein said groin covering panel comprises a first-fastener and a second-fastener located on a top portion and a bottom portion, respectively of said groin covering panel;
wherein said leg openings are formed by fastening a middle-fastener of said securing strap to said first-fastener and fastening a first-end-fastener of said securing strap and a second-end fastener of said securing strap on opposing sides of said second-fastener located at said bottom portion of said groin covering panel; and
wherein said protective barrier system provides an absorbent article and protective barrier for absorbing excess fluids during said sexual activities.

2. The protective barrier system of claim 1, wherein said securing strap is elasticized.

3. The protective barrier system of claim 1, wherein said groin covering panel comprises at least one wicking material.

4. The protective barrier system of claim 1, wherein said fastening members comprise hook and loop fasteners.

5. The protective barrier system of claim 4, wherein said first-fastener and said second-fastener are located on said groin covering panel comprise female-fasteners of said hook and loop fasteners.

6. The protective barrier system of claim 5, wherein said first-end-fastener, said middle-fastener, and said second-end fastener of said securing strap comprise male-fasteners of said hook and loop fasteners for mating with said female-fasteners located on said groin covering panel.

7. The protective barrier system of claim 1, wherein said securing strap when fastened to said groin covering panel creates said leg openings which are configured to receive legs of said user-wearer and secure said groin covering panel to said user-wearer.

8. The protective barrier system of claim 1, wherein said aperture comprises an elastic collar.

9. The protective barrier system of claim 1, wherein said first-end-fastener and said second-end fastener comprise a smaller surface area than said middle-fastener, said first-fastener, and said second-fastener.

10. The protective barrier system of claim 9, wherein said second-fastener comprises a surface area which is configured to receive both said first-end-fastener and said second-end fastener.

11. The protective barrier system of claim 1, wherein said groin covering panel comprises a substantially rectangular profile.

12. The protective barrier system of claim 11, wherein said groin covering panel comprises contoured edges which are configured to contour to legs of said user-wearer.

13. The protective barrier system of claim 1, wherein said groin covering panel comprises a planar sheet.

14. The protective barrier system of claim 1, wherein said aperture is located at a center of said groin covering panel.

15. The protective barrier system of claim 1, wherein said first-fastener and said second-fastener are located on a back side of said groin covering panel.

16. The protective barrier system of claim 15, wherein said back side of said groin covering panel is configured to rest against a pelvic region of said user-wearer.

17. A protective barrier system comprising:
a protective barrier assembly including;
a groin covering panel;
an aperture;
a securing strap; and
fastening members;
wherein said protective barrier system comprises said protective barrier assembly;
wherein said protective barrier assembly comprises said groin covering panel, said securing strap, and said fastening members in functional combination;
wherein said groin covering panel is formed of flexible material with leg openings, and is of a size and shape configured to cover a pelvic region of a user-wearer;
wherein said groin covering panel comprises a substantially rectangular profile;
wherein said groin covering panel comprises contoured edges which are configured to contour to legs of said user-wearer;
wherein said groin covering panel comprises a planar sheet;
wherein said groin covering panel comprises at least one wicking material;
wherein said groin covering panel comprises said aperture configured to allow genitals to be accessed for sexual activities;
wherein said aperture comprises an elastic collar;
wherein said aperture is located at a center of said groin covering panel;
wherein said securing strap comprises said fastening members located at a first-end, a middle, and a second-end of said securing strap;
wherein said fastening members comprise hook and loop fasteners;
wherein said first-fastener and said second-fastener are located on said groin covering panel comprise female-fasteners of said hook and loop fasteners;
wherein said first-fastener and said second-fastener are located on a back side of said groin covering panel;
wherein said back side of said groin covering panel is configured to rest against a pelvic region of said user-wearer;
wherein said first-end-fastener, said middle-fastener, and said second-end fastener of said securing strap comprise male-fasteners of said hook and loop fasteners for mating with said female-fasteners located on said groin covering panel;
wherein said first-end-fastener and said second-end fastener comprise a smaller surface area than said middle-fastener, said first-fastener, and said second-fastener;
wherein said second-fastener comprises a surface area which is configured to receive both said first-end-fastener and said second-end fastener;
wherein said securing strap when fastened to said groin covering panel creates said leg openings which are configured to receive legs of said user-wearer and secure said groin covering panel to said user-wearer;
wherein said groin covering panel comprises a first-fastener and a second-fastener located on a top portion and a bottom portion, respectively of said groin covering panel;
wherein said leg openings are formed by fastening a middle-fastener of said securing strap to said first-fastener and fastening a first-end-fastener of said securing strap and a second-end fastener of said securing strap on opposing sides of said second-fastener located at said bottom portion of said groin covering panel;
wherein said securing strap is elasticized; and
wherein said protective barrier system provides an absorbent article and protective barrier for absorbing excess fluids during said sexual activities.
